(19) United States
(12) Patent Application Publication
(10) Pub. No.: US 2008/0127363 A1
(43) Pub. Date: May 29, 2008

(54) Flowering Time-Controlling Gene Cog2 Isolated from Arabidopsis Thaliana

(76) Inventors: Hong Gil Nam, Kyungsangbuk-do (KR); Jeong Sik Kim, Kyungsangbuk-do (KR); Don Ha Park, Columbus, OH (US); Pyung Ok Lim, Kyungsangbuk-do (KR)

Correspondence Address: SUGHRUE MION, PLLC, 2100 PENNSYLVANIA AVENUE, N.W., SUITE 800, WASHINGTON, DC, 20037, US

(21) Appl. No.: 10/594,578

(22) PCT Filed: Mar. 29, 2005
(86) PCT No.: PCT/KR05/00910

§ 371 Date: Sep. 27, 2006

(30) Foreign Application Priority Data
Mar. 29, 2004 (KR) 10 2004-0021216

(51) Int. Cl. C12N 15/87; C07K 16/00; C07H 21/04; C12N 5/04; A01H 1/00

(52) U.S. Cl. 800/278; 530/350; 536/23.6; 435/419; 800/298

(57) ABSTRACT

A flowering time-controlling gene, COG2, isolated from Arabidopsis thaliana, and a protein expressed therefrom delay the flowering time of a plant and, accordingly, are useful for improving a flowering-associated character of a plant and for the identification of other flowering-associated genes or proteins.

### FIELD OF THE INVENTION

The present invention relates to a flowering time controlling gene, COG2, isolated from Arabidopsis thaliana, a protein encoded therein, and a method for controlling the flowering time of a plant comprising the step of transforming the plant with the COG2 gene.

### BACKGROUND OF THE INVENTION

The flowering time of plants varies depending on temperature, duration of daylight (i.e., photoperiod), or both. Based on a relationship between the photoperiod and the flowering time, plants are largely divided into three classes; long daylight plants which flowers under long photoperiod, short daylight plants which flowers under short photoperiod, and day-neutral plants whose flowering time does not depend on the photoperiod. It has been reported that such flowering characteristics are controlled by several genes (Yaron et al., The Plant Cell 10: 1973-1989 (1998)).

The flowering time has been an important subject of the classic breeding in agriculture, because it is a major factor in determining crop's market value, variety, etc. The breeding method used in classic breeding is typically a cross-breeding method, which requires repeated steps of breeding until the unnecessary characters are removed and a desired character is introduced. However, the cross-breeding method has problems in that it takes a long, painstaking period of 5 to 20 years to obtain the desired cross-breeding variety, and moreover, the resulting varieties may be found to have other recessive character or sensitivity to pathogens only after it is made available to the public. Recently, with the aid of genetic engineering technologies, it is possible to isolate genes related to the control of flowering time and to utilize the genes in breeding with appropriate manipulation thereof. Consequently, it is expected that new breed varieties having well-controlled flowering time can be developed (Ove Nilson et al., Current Opinion in Biotechnology 8: 195-199 (1997)).

It has been reported that the mechanisms of controlling the flowering time in plants are quite complicated, involving interactions among various genes (Alon Samach et al., Coupland BioEssays 22: 38-47 (2000)).

Recently, a genome project of Arabidopsis thaliana, a model species of dicotyledons, was completed with the aid of molecular biology technologies, and the amino sequences of numerous genes thereof were published (Arabidopsis genome initiative, Nature 408, 796-815 (2000)). Further, it became possible to predict the functions of about 30% of the genes by comparing its nucleotide sequences with those from other model systems. However, such result is useful only in predicting the functions of the genes at a molecular level, but not in understanding the exact functions of the genes in a living organism. For instance, the result of the genome project exhibited that a large portion of Arabidopsis thaliana genes have very similar or overlapped sequences, but it was reported that these genes actually function differently from each other (Gualerti G., et al., Plant Cell 14: 1253-63 (2002)). Therefore, it has been required to screen a gene having an amino sequence similar to that of a gene having a well-established function, and analyze the function of the screened gene by employing a transformant overexpressing the gene.

The flowering of Arabidopsis thaliana is stimulated under a long daylight condition, and it has been found that there are three pathways controlling the flowering of Arabidopsis thaliana. The first pathway is an autonomous pathway, wherein the flowering is controlled regardless of the duration of daylight, and the genes such as LD, PGM1, FY, FCA, FPA, and FLD are involved in this pathway. The second pathway is a photoperiodic pathway, wherein the flowering of plants is controlled by sensing the duration of daylight, and the genes such as ELF3, CAM1, GI, CO, FWA, FT, and FE are involved in this pathway. The third pathway is vernalization pathway, wherein the flowering is controlled by temperature, and the genes including VRN1, VRN2, FRI, and FLC are involved in this pathway.

A molecular biological approach to Arabidopsis thaliana has been easy due to the completion of genomic and physical mapping of their five chromosomes and recently accomplished nucleotide sequencing of the chromosomes. In Arabidopsis thaliana, a flowering-inducing signal is produced due to environmental factors such as a photoperiod or intrinsic factors. The signal induces a phase transition from vegetative growth to reproductive growth, which stimulates flowering. Accordingly, the number of leaves during the bolting time, i.e., a period when the length of a flower stalk becomes approximately 1 to 3 cm, can be used as an indicator for the flowering time. In addition, Arabidopsis thaliana is advantageous as a tool for studying the accurate in vivo function of a gene because a transformant of Arabidopsis thaliana, which is transformed with the gene, can be easily prepared by employing Agrobacterium.

The present inventors have discovered a novel flowering time-controlling gene, COG2, from Arabidopsis thaliana, and have achieved the present invention by confirming that the flowing time of an Arabidopsis thaliana transformed with COG2 gene can be controlled by varying the photoperiod.

### SUMMARY OF THE INVENTION

Accordingly, it is an object of the present invention to provide a gene controlling the flowering time and a protein encoded therein.

It is another object of the present invention to provide an expression vector comprising the flowering time-controlling gene and a plant transformed with the vector.

It is a further object of the present invention to provide a method for controlling the flowering time of a plant using the gene.

It is a still further object of the present invention to provide a method for identifying other flowering time-controlling gene or protein using the gene or protein.

In accordance with one aspect of the present invention, there is provided a flowering time-controlling protein, COG2, which is isolated from Arabidopsis thaliana and has an amino acid sequence of SEQ ID NO: 2, and a DNA encoding the protein.

In accordance with another aspect of the present invention, there is provided an expression vector comprising a DNA encoding COG2 and Arabidopsis thaliana transformants, COG2OX-9 and COG2OX-13, transformed with the expression vector.

In accordance with a further aspect of the present invention, there is provided a method for controlling the flowering time of a plant, which comprises transforming the plant with a DNA encoding the COG2 protein.

In accordance with a still further aspect of the present invention, there is provided a method for identifying a flowering time-controlling gene or protein, which comprises using the COG2 protein or a DNA encoding same as a probe.

### BRIEF DESCRIPTION OF THE DRAWINGS

The above and other objects and features of the present invention will become apparent from the following description of the invention, when taken in conjunction with the accompanying drawings, which respectively show:

FIG. 1: the result of Northern blotting for COG2 gene expressed in transformants COG2OX-9 and COG2OX-13, as compared with that of wild-type Arabidopsis thaliana (Columbia);

FIG. 2: the flowering time and the number of leaves of the transformants COG2OX-9 and COG2OX-13, and Columbia, under a long daylight condition; and

FIG. 3: the flowering time and the number of leaves of the transformants COG2OX-9 and COG2OX-13, and Columbia, under a short daylight condition.

### DETAILED DESCRIPTION OF THE INVENTION

The inventive gene controlling flowering time, COG2, isolated from Arabidopsis thaliana, includes an opening reading frame (ORF) having a nucleotide sequence of SEQ ID NO: 1 consisting of 513 bp, the ORE comprising only one exon.

However, in consideration of the degeneracy of codons and the preferred codons in a specific organism wherein COG2 gene is to be expressed, various changes and modifications of the nucleotide sequence of SEQ ID NO: 1 may be made, e.g., in the coding area thereof without adversely altering the amino acid sequence of the flowering time-controlling protein expressed therefrom, or in the non-coding area without adversely affecting the expression of the gene. Therefore, the present invention also includes, in its scope, a polynucleotide having substantially the same nucleotide sequence as the inventive COG2 gene of SEQ ID NO: 1, and a fragment thereof. As used herein, “substantially the same polynucleotide” refers to a polynucleotide encoding a protein having the amino acid sequence of SEQ ID NO: 2, whose nucleotide sequence shows 80% or more, preferably 90% or more, most preferably 95% or more homology to the nucleotide sequence of SEQ ID NO: 1.

The protein expressed from the COG2 gene of the present invention consists of 170 amino acids and has the amino acid sequence of SEQ ID NO: 2. The molecular weight of this protein is about 19 kDa. According to the result of an analysis of the protein using a computer program for the prediction of protein localization (PSORT; http://psort.nibb.ac.jp), it seems that the protein functions in the nucleus. Moreover, the amino acid sequencing of the protein reveals that an amino acid sequence known as DOF (DNA binding with One Finger) domain, which is commonly shared among the transcription factors of plants, is conserved in the protein (aa 60-110 of SEQ ID NO: 2). The genes containing DOF domain exist only in plants and known as transcription factors which involves in various physiological phenomena such as activations of a gene associated with photosynthesis, a gene responsible for a seed storing protein, a plant oncogene, a gene induced by plant hormones, a stress inducing gene, etc. (Yanagisawa et al., Trends in Plant Science, 1: 213-214 (1996)).

However, various substitution, addition and/or deletion of the amino acid residues of protein may be performed without adversely affecting the protein's function. Further, a portion of the protein may be used when a specific purpose is to be fulfilled. These modified amino acids and fragments thereof are also included in the scope of the present invention. Therefore, the present invention includes, in its scope, a polypeptide having substantially the same amino acid sequence as the protein derived from the COG2 gene of the present invention and a fragment thereof. As used herein, “substantially the same polypeptide” refers to a polypeptide whose amino acid sequence shows 80% or more, preferably 90% or more, most preferably 95% or more homology to the amino acid sequence of SEQ ID NO: 2.

The COG2 gene and protein of the present invention are useful in improving flowering-associated characters of plants and for identifying flowering-associated genes and proteins in other plants. They can be used as molecular probes in a method for identifying genes responsible for flowering in plants, which screens a substance that bind to the gene or that inhibit or activate the expression of the gene. The identification of a gene or protein can be carried out by various conventional methods including DNA chip, protein chip, polymerase chain reaction (PCR), Northern blotting, Southern blotting, Western blotting, enzyme-linked immunosorbent assay (ELISA), 2-D gel analysis, etc.

Meanwhile, the COG2 gene or protein of the present invention can be isolated from Arabidopsis thaliana or synthesized using a conventional DNA or peptide synthesis method. Further, the gene thus prepared may be inserted to a conventional vector to obtain an expression vector, which may, in turn, be introduced into a suitable host, e.g., a microorganism such as Agrobacterium.

Specifically, in a preferred embodiment of the present invention, COG2 gene is inserted into an appropriate vector such as pCAMBIA 3301 binary vector to obtain an expression vector, transforming an Agrobacterium with the expression vector, and transforming the wild-type Arabidopsis thaliana with the transformed Agrobacterium to obtain transformants COG2OX-9 and COG2OX-13 exhibiting flowering delaying phenotypes under a long daylight condition.

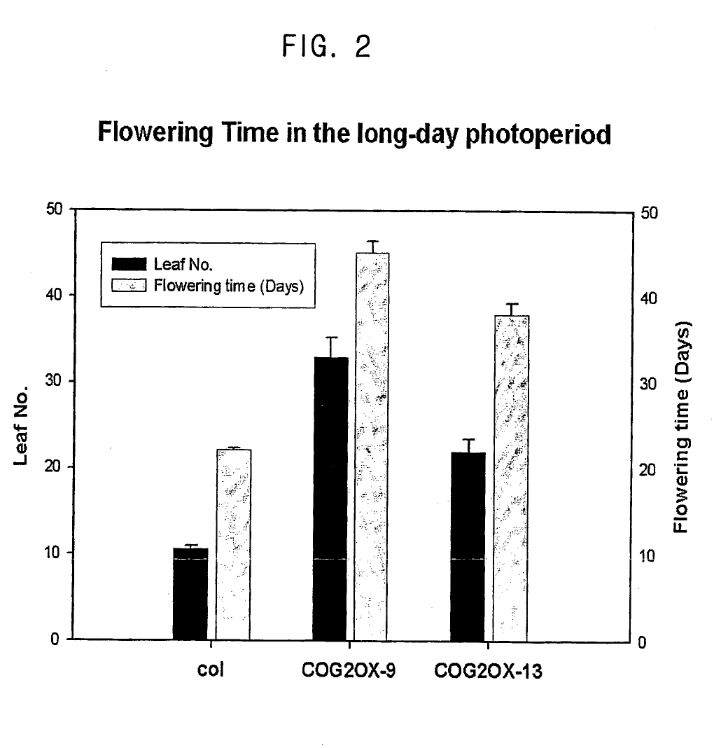

The Arabidopsis thaliana transformants COG2OX-9 and COG2OX-13 show more delayed flowering times and more leaves in the flowering time than the wild-type Arabidopsis thaliana, Columbia, under a long daylight condition (see FIG. 2). Consequently, the inventive COG2 gene can be used for controlling the flowering time of long daylight plants such as rice, as well as Arabidopsis thaliana.

The present invention also provides a method for controlling the flowering time of a plant using the COG2 gene. The flowering time of a plant can be delayed by transforming the plant with an expression vector containing the COG2 gene and overexpressing the COG2 gene. Representative plants of which flowering time can be controlled by the method of the present invention include: food crops such as rice, wheat, barley, corn, bean, potato, red bean, oat and millet; vegetable crops such as Arabidopsis, Chinese cabbage, radish, red pepper, strawberry, tomato, watermelon, cucumber, cabbage, melon, squash, stone-leek, onion, and carrot; special use crops such as ginseng, tobacco, cotton, sesame, sugar cane, sugar beet, wild sesame, peanut, and rape; fruits such as apple, pear, date, peach, western Actinidia, grape, orange, persimmon, plum, apricot and banana; flowers such as rose, gladiolus, gerbera, carnation, mum, lily and tulip; fodder crops such as ryegrass, red clover, orchard grass, alfalfa, tall fescue and perennial ryegrass. Especially, the method of the present invention would be commercially valuable when it is applied to green vegetables such as lettuce or spinach of which green leaves rapidly age upon flowering.

The following Examples are intended to further illustrate the present invention without limiting its scope.

### EXAMPLE 1

### Isolation of Flowering Time Controlling Gene, COG2 from Arabidopsis thaliana and Sequencing of the Gene

The genomic DNA of Arabidopsis thaliana was isolated using CTAB (cetyltrimethylammoniumbromide, Sigma, USA). Based on NCBI database (http://www.ncbi.nih.nlm.gov/blast/), a gene having a sequence similar to that of a known flowering time controlling gene of Arabidopsis thaliana, COG (PCT WO 03/018626), was screened from the isolated genomic DNA.

The following reactions were carried out to isolate the screened gene from the Arabidopsis thaliana genomic DNA. 10 ng of Arabidopsis thaliana genomic DNA was mixed with 1 unit of ExTaq polymerase (Takara, JP), 5 ul of 10× Reaction buffer, 20 pmol of a synthetic forward primer of SEQ ID NO: 5 having NcoI site, 20 pmol of a synthetic reverse primer of SEQ ID NO: 6 having BstEII site, and 2.5 mM of dNTP. Then, polymerase chain reaction (PCR) was carried out using GeneAmp 9600 (PerkinElmer) under the conditions of initial denaturation at 95° C. for 3 min; 35 cycles of denaturation at 94° C. for 15 sec, annealing at 53° C. for 30 sec and extension at 72° C. for 50 sec; and final extension at 72° C. for 7 min. The resulting PCR products were subjected to 1% agarose gel electrophoresis at 100 V for 1 hour, a DNA band containing COG2 gene was cut out, and COG2 gene was eluted therefrom with a gel elution kit (Quiagen). Then, the gene was subcloned into T vector using GEM T Easy Vector Kit (Promega, US).

The sequence of the cloned gene was analyzed and the result revealed that the gene has the nucleotide sequence of SEQ ID NO: 1 and encodes a protein having the amino acid sequence of SEQ ID NO: 2, as analyzed with Translation tool (http://kr.expasy.org/tools/dna.html). Further, the nucleotide sequences of the cloned gene and the known flowering time-controlling gene, COG, and the amino acid sequences of proteins encoded therein were compared with each other, respectively.

The gene having the nucleotide sequence of SEQ ID NO: 1 showed 78% homology with the nucleotide sequence of the COG gene(SEQ ID NO: 3) and the protein having the amino acid sequence of SEQ ID NO: 2 displays 77% homology and 82% similarity with the amino acid sequence of COG protein (SEQ ID NO: 4). The gene isolated from Arabidopsis thaliana was designated “COG2 gene”. The protein expressed from the COG2 gene, i.e., COG2 protein, consisted of 170 amino acids, and its molecular weight was 19 kDa. By using a computer program for the prediction of protein localization (PSORT; http://psort.nibb.ac.jp/), the COG2 protein was found to be present inside the nucleus. Accordingly, it was assumed that, similar to COG protein, the COG2 protein functions in the nucleus. Further, an amino acid sequence known as DOF (DNA binding with One Finger) domain, which is commonly shared among the transcription factors, was also observed in the COG2 protein (amino acids 60˜110 of the SEQ ID NO: 2).

### EXAMPLE 2

### Synthesis of an Expression Vector and a Transformant Comprising COG2 Gene

The recombinant vector prepared in Example 1, which contains COG2 gene cloned into T easy vector, and the binary vector pCAMBIA 3301 (Cambia, USA) were respectively treated with 2 μl (10 unit/μl) of restriction enzymes, NcoI and BstEII at 37° C. for 4 hr. Then, the resulting products were subjected to 1% agarose gel electrophoresis, and a 0.54 kb DNA fragment containing the COG2 gene and a 9.26 kb DNA fragment corresponding to the NcoI/BstEII fragment of pCAMBIA 3301 vector were eluted from the gel with a gel elution kit (Quiagen). The DNA fragments were mixed together and treated with 1 μl (1 unit/μl) of T4 ligase (Roche) at 16° C. for 6 hr to obtain a vector containing COG2 gene. An Agrobacterium (Agrobacterium tumefaciens ABI strain, USA, Amasino laboratory) was transformed with the vector according to an electric shock method. The resulting transformant was further utilized to transform the wild-type Arabidopsis thaliana, Columbia (Arabidopsis stock center, USA) according to floral dipping methods (Clough et al., Plant J., 16(6): 735-743). After collecting seeds from the transformed Arabidopsis thaliana, 1×10⁵ seeds were sowed on 10 pots each of 15 cm×15 cm size, and treated with 0.03% basta (Korea) herbicide. The survived seeds were selected and cultivated in an artificial culture system under 16L/8D condition. Consequently, approximately 30% of the COG2 transformants exhibited flowering delaying phenotypes under the long daylight condition, although some individual variations occurred. Two of the transformants that displayed the most delayed flowering times were selected and designated COG2OX-9 and COG2OX-3, respectively.

### EXAMPLE 3

### Northern Blotting Analysis Confirming the COG2 Gene Expression in the Transformants

In order to examine whether the COG2 gene is overexpressed in the transformants, the COG2 gene expressions were studied using the Northern blotting method.

Total RNAs were extracted from each of the tissues of the wild-type Arabidopsis thaliana, Columbia and the transformants, COG2OX-9 and COG2OX-13 prepared in Example 2 using Tri-Reagent Kit (molecular research center, USA). 30 μg of the RNA was subjected to 1.2% formaldehyde-agarose gel and the separated RNA bands were transferred to a nylon membrane (Hybond N⁺, Amersham, USA) by employing a vacuum transport system (USA). The nylon membrane was exposed to UV-light on a UV-crosslinker (Stratagene) and dried at 65° C. for 1 hr.

In order to prepare a radio-labeled probe for detecting the COG2 gene, the recombinant vector prepared in Example 1 was cut with EcoRI to obtain the COG2 gene, which was then radio-labeled by employing a random priming kit (Amersham pharmacia, USA) and 5 μl of dCTP (1 mCi/μl, Amersham pharmacia) according to the manufacturer's instruction. One of actin genes, ACT8 was used to compare its expression level with that of COG2 gene. A DNA coding for actin was amplified according to the PCR method described in Example 1, by employing a forward primer of SEQ ID NO: 7 and a reverse primer of SEQ ID NO: 8. The PCR product was subjected to the radio-labeling process as described above, and the resulting labeled DNA was used as a probe for detecting actin.

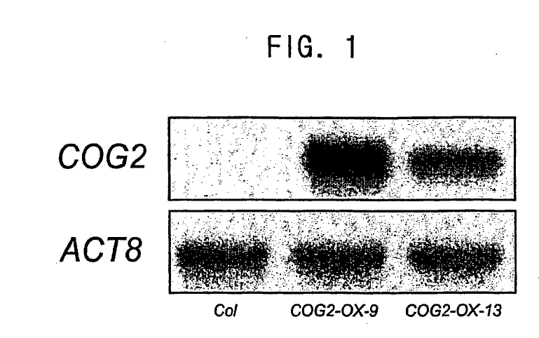

The dried membrane was treated with the 20 ul of radio-labeled probes and was subjected to hybridization reaction with Church and Gilbert solution (25 mM sodium phosphate, 1 mM EDTA, 7% SDS, and 1% BSA) at 65° C. for 16 hr. Then, the membrane was washed with a washing buffer containing 2×SSC and 0.2% SDS at 65° C. for 10 min, followed by an additional wash with a washing buffer containing 1×SSC and 0.1% SDS for 10 min and a final wash with a buffer containing 0.1×SSC and 0.1% SDS. The nylon membrane was exposed to BAS film for 2 days and analyzed using a phosphorimager (BAS2000, Fuji, Japan). The result is shown in FIG. 1.

As can be seen from FIG. 1, COG mRNA expression is extremely low in Columbia while it is high in COG2OX-9 and COG2OX-13. Specifically, the COG2 mRNA expression level was higher in COG2OX-9 than in COG2OX-13.

### EXAMPLE 4

### Comparison of the Flowering Times of Columbia and the Arabidopsis thaliana Transformants

In order to examine the degree of flowering time delay in the Arabidopsis thaliana transformants, COG2OX-9 and COG2OX-13 relative to wild-type Arabidopsis thaliana, a homo line that showed uniform genotype was selected. Seeds of wild-type Columbia (Arabidopsis stock center, USA) and two transformants were immersed in water at 4° C. in the dark for 3 to 5 days to adjust the germination time of the seeds identical. The seeds were sowed at regular intervals in pots, and the pots were covered with a wrap for about 7 days to keep the moisture. Once the seeds germinated, the pots were placed under two different conditions, i.e., a long daylight condition of 16L/8D and a short daylight condition of 8L/16D. The number of days from germination to flowering time, and the number of rosette leaves when the length of the flower stalk became about 3 cm were recorded.

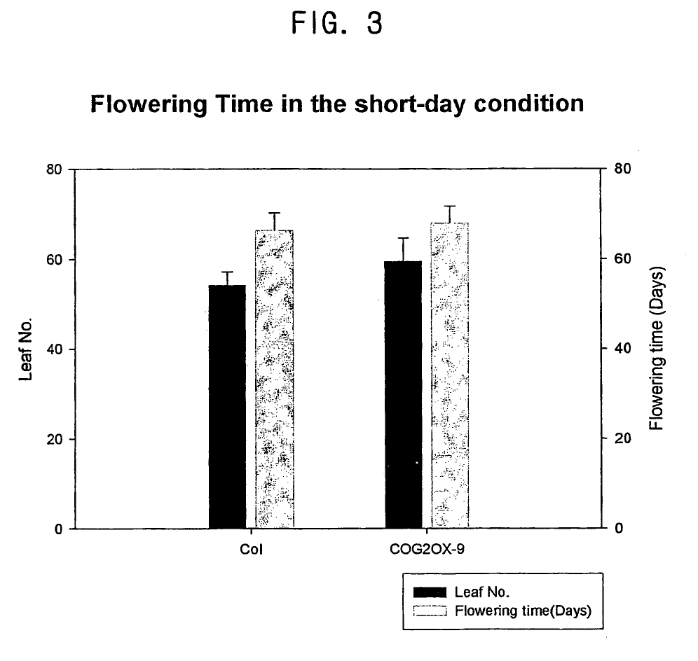

FIGS. 2 and 3 compare the flowering times between Columbia and the transformants under long daylight and short daylight conditions, respectively. As can be seen in FIG. 2, under a long daylight condition, Columbia flowered on day 22.5, while the flower stalk of transformants COG2OX-9 and COG2OX-13 appeared only on day 44 and 38, respectively. Moreover, transformants COG2OX-9 and COG2OX-13 had greater number of rosette leaves at the flowering time than Columbia. This result indicates that the transformants would flower in late autumn. Meanwhile, as can be seen from FIG. 3, the flowering time and the number of rosette leaves of transformant COG2OX-9 observed under the short daylight condition were similar to those of Columbia.

While the invention has been described with respect to the above specific embodiments, it should be recognized that various modifications and changes may be made to the invention by those skilled in the art which also fall within the scope of the invention as defined by the appended claims.

What is claimed is:

1. A flowering time controlling protein, COG2, having the amino acid sequence of SEQ ID NO: 2.

2. A DNA encoding the COG2 protein of claim 1.

3. The DNA of claim 2, which has the nucleotide sequence of SEQ ID NO: 1.

4. An expression vector comprising the DNA of claim 2.

5. A microorganism transformed by the expression vector of claim 4.

6. A plant transformed by the expression vector of claim 4.

7. A method for controlling the flowering time of a plant, which comprises transforming the plant with the DNA of claim 2.

8. The method of claim 7, wherein the flowering time of a plant is controlled by transforming the plant with an expression vector comprising the DNA of claim 2 and overexpressing COG2 gene in the transformed plant.

9. The method of claim 8, wherein the plant is selected from the group consisting of: food crops including rice, wheat, barley, corn, bean, potato, red bean, oat and millet; vegetable crops including Arabidopsis, Chinese cabbage, radish, red pepper, strawberry, tomato, watermelon, cucumber, cabbage, melon, squash, stone-leek, onion, and carrot; special crops including ginseng, tobacco, cotton, sesame, sugar cane, sugar beet, wild sesame, peanut, and rape; fruits including apple, pear, date, peach, western Actinidia, grape, orange, persimmon, plum, apricot and banana; flowers including rose, gladiolus, gerbera, carnation, mum, lily and tulip; fodder crops including ryegrass, red clover, orchard grass, alfalfa, tall fescue and perennial ryegrass.


SEQUENCE LISTING (8 sequences)

SEQ ID NO: 1 - DNA, 513 bp, Arabidopsis thaliana
atggcgactcaagattctcaagggattaaactctttggcaaaactattgcatttaacactcgaacaataaaaaatgaagaagagacacacccgccggagcaagaagccacaatagccgttagatcatcatcatcatcggatctgacggccgagaagcgtccggataagatcatagcatgtccaagatgcaagagcatggagacaaagttctgttacttcaacaactacaacgttaatcagcctcgacacttttgtaaaggctgccaccgttactggaccgccggtggtgcactccggaacgttcccgtcggcgccggtcgtcggaagtccaaaccacctggtcgtgtcgtggttggtatgcttggagatggaaatggtgttcgccaagtcgagcttataaatggcttgctcgttgaggagtggcagcatgccgcagccgcagctcacggtagtttccggcatgattttcccatgaagcggctccggtgttactccgacggtcaatcgtgctga

SEQ ID NO: 2 - protein, 170 aa, Arabidopsis thaliana
  SIGNAL (107)..(111): Nucleus localization signal
MATQDSQGIKLFGKTIAFNTRTIKNEEETHPPEQEATIAVRSSSSSDLTAEKRPDKIIACPRCKSMETKFCYFNNYNVNQPRHFCKGCHRYWTAGGALRNVPVGAGRRKSKPPGRVVVGMLGDGNGVRQVELINGLLVEEWQHAAAAAHGSFRHDFPMKRLRCYSDGQSC

SEQ ID NO: 3 - DNA, 528 bp, Arabidopsis thaliana
atggcgacccaagattctcaagggattaaactctttggcaaaaccataacattcaacgccaacatcacacagacgataaaaaaagaagagcagcaacaacaacaacagccagagctacaagcaacaacagccgttagatcaccctcatcggatctgacggctgagaagcgtccagacaagatcataccatgtccgagatgcaagagcatggagactaagttttgttacttcaacaactacaacgttaatcaaccaagacacttctgcaaaggttgtcaacgttactggaccgccggtggagctctccggaatgttcccgtcggtgccggtcgtcggaagtcaaaacctcccggacgtgtcggtgggttcgctgagttgcttggagctgcgactggagctgttgatcaggtcgagctagatgctttgctagtggaagagtggagagctgctacggcgtctcacggtggtttccggcatgattttccggtgaagaggctccgttgttacaccgatggtcaatcttgttaa

SEQ ID NO: 4 - protein, 175 aa, Arabidopsis thaliana
MATQDSQGIKLFGKTITFNANITQTIKKEEQQQQQQPELQATTAVRSPSSDLTAEKRPDKIIPCPRCKSMETKFCYFNNYNVNQPRHFCKGCQRYWTAGGALRNVPVGAGRRKSKPPGRVGGFAELLGAATGAVDQVELDALLVEEWRAATASHGGFRHDFPVKRLRCYTDGQSC

SEQ ID NO: 5 - DNA, 25 bp, Artificial Sequence
  forward primer specific for COG2
ccatggccattctctgcacgttttc

SEQ ID NO: 6 - DNA, 23 bp, Artificial Sequence
  reverse primer specific for COG2
ggtnacctcagcacgattgaccg

SEQ ID NO: 7 - DNA, 24 bp, Artificial Sequence
  forward primer specific for ACT8
aatcagatgtggatctctaaggca

SEQ ID NO: 8 - DNA, 24 bp, Artificial Sequence
  reverse primer specific for ACT8
tccgagtttgaagaggctacaaac